(19) United States
(12) Patent Application Publication
(10) Pub. No.: US 2020/0203017 A1
(43) Pub. Date: Jun. 25, 2020

(54) SYSTEMS AND METHODS OF PREDICTION OF INJURY RISK WITH A TRAINING REGIME

(71) Applicant: ALERTE DIGITAL SPORT PTY LTD, Perth, Western Australia (AU)

(72) Inventors: James Calham DOWER, Jan Juc, Victoria (AU); Razali Bin MOHAMAD, Melbourne, Victoria (AU)

(21) Appl. No.: 16/620,602

(22) PCT Filed: Jun. 8, 2018
(86) PCT No.: PCT/AU2018/050571

§ 371 Date: Dec. 9, 2019

(30) Foreign Application Priority Data
Jun. 9, 2017 (AU) 2017902212

(51) Int. Cl. G16H 50/30; G16H 20/30; G16H 10/60; G16H 50/50; G16H 50/70; G06N 3/08

(52) U.S. Cl. CPC G16H 50/30; G16H 20/30; G06N 3/084; G16H 50/50; G16H 50/70; G16H 10/60

(57) ABSTRACT

Systems and methods for forming a simulation of a proposed training regime and providing a probability risk factor prediction in respect of one or more participants participating in said proposed training regime; the system comprising: a user input means adapted for accepting user input regarding: a proposed training regime; and training data regarding one or more participants of the proposed training regime; a storage means adapted for storing records regarding: said historical training data regarding one or more past training activity; historical participant data regarding one or more participants of the proposed training regime; and historical actual participant outcomes from said past training activity; and a system controller comprising a processor adapted to: receive user input regarding said proposed training regime and proposed participants; perform calculations to analyse said user input in conjunction with said historical training data and said historical participant outcomes; perform calculations to compute a probability risk factor prediction for each proposed participant as a result of participating in said proposed training regime; and display a visual representation of said computed probability risk factor prediction on a user interface.

### FIELD OF THE INVENTION

The present invention relates to artificial neural networks for predicting participant outcomes from a proposed physical exercise training regime and in particular to systems and methods system for forming a simulation of a proposed training regime and providing a probability risk factor prediction in respect of one or more participants participating in the proposed training regime.

The invention has been developed primarily for use in methods and systems for predicting participant outcomes from a proposed physical exercise training regime in relation to training for sporting events and will be described hereinafter with reference to this application. However, it will be appreciated that the invention is not limited to this particular field of use.

### BACKGROUND

Any discussion of the background art throughout the specification should in no way be considered as an admission that such background art is prior art nor that such background art is widely known or forms part of the common general knowledge in the field in Australia or worldwide.

Artificial intelligence (AI) is the ability of a computer program or a machine to think and learn, and in general usage, the term AI refers to the ability of a computer or machine to mimic human cognition. An ideal artificial intelligence is a flexible machine which perceives its environment (e.g. examples of a particular situation and/or outcomes from particular situations) and automatically takes actions to maximise its success in achieving a particular defined goal. AI has been applied in a variety of fields including understanding human speech, competing at a high level in strategic game systems such as Chess or Go, self-driving cars and interpreting complex data and providing an actionable output. Artificial neural networks (ANNs) or connectionist systems are a computational model used in machine learning, computer science and other research disciplines and a known as a particular type of computer architecture used to realise an artificial intelligence system. ANNs are based on a large collection of connected simple units called artificial neurons, loosely analogous to axons in a biological brain. Connections between ‘neurons’ carry an activation signal of varying strength or weight. If the combined incoming signals are strong enough, the neuron becomes activated and the signal travels to other neurons connected to it. Such systems can be trained from examples, rather than explicitly programmed, and excel in areas where the solution or feature detection is difficult to express in a traditional computer program. Like other machine learning methods, neural networks have been used to solve a wide variety of tasks, like computer vision and speech recognition, that are difficult to solve using ordinary rule-based programming.

For example, the classification of images into object categories is an example of an AI application i.e. Is this a picture of a car? Pixels are used as inputs in to the AI. Known image categories (i.e. a labelled data set), consists of a set of images that have been manually labelled and categorised. These examples are used to calibrate (or teach) the AI. Pre-processing techniques are used to assist the AI. Examples in pre-processing include normalisation of pixel values from a range of 0-255->0-1 or −1,1. This normalisation process assists in the optimisation of the neural network during training. Another form of pre-processing would be resizing of an image. It may not be feasible to have a very large, high detailed image which requires a larger amount of computation. The same result could be achieved by subsampling the image in to a smaller form.

Another example of an AI system is one used for voice recognition by a computer system which includes training an AI to recognise words from verbal speech. The system is typically trained by recording (or ‘listening to’) verbal speech by a person that is reading a known set of words: phrases, sentences, paragraphs etc. This recorded speech becomes the labelled data set. Examples of pre-processing to assist the AI would include filtering of noise, normalisation of frequency and similar audio processing actions. Such AI systems however, typically require large computing resources that make them impractical for personal use. Furthermore, AI systems generally suffer from significant imbalances between data describing different classes of event e.g. injury vs. no injury—in this particular case there is much more data describing an event where no injury has occurred compared with events in which an injury occurred—and the system must be designed to account for such imbalances. Also, AI systems are highly application specific and rely on being trained with specific data to achieve a desired output. Accordingly, a particular AI system that can achieve good results in one field may be completely incompatible if applied in a different field and thus the quality of the output from the AI is highly dependent upon the input data type and format thus requiring the input data to be properly formulated based on the problem to be solved and pre-processed in a highly specific ways that can be interpreted by the AI for the desired outcome.

### SUMMARY

It is an object of the present invention to overcome or ameliorate at least one or more of the disadvantages of the prior art, or to provide a useful alternative.

One embodiment provides a computer program product for performing a method as described herein.

One embodiment provides a non-transitive carrier medium for carrying computer executable code that, when executed on a processor, causes the processor to perform a method as described herein.

One embodiment provides a system configured for performing a method as described herein.

According to a first aspect of the present invention, there is provided a system for forming a simulation of a proposed training regime and providing a probability risk factor prediction in respect of one or more participants participating in the proposed training regime. The system may comprise a user input means adapted for accepting user input. The user input may comprise data regarding a proposed training regime. The user input may further comprise data regarding training data regarding one or more participants of the proposed training regime. The system may further comprise a storage means. The storage means may be adapted for storing records regarding the historical training data regarding one or more past training activity. The records may further comprise data regarding historical participant data regarding one or more participants of the proposed training regime. The records may further comprise data regarding historical actual participant outcomes from the past training activity. The system may further comprise a system controller comprising a processor. The processor may be adapted to receive user input regarding the proposed training regime and proposed participants. The processor may be further adapted to perform calculations to analyse the user input in conjunction with the historical training data and the historical participant outcomes. The processor may be further adapted to perform calculations to compute a probability risk factor prediction for each proposed participant as a result of participating in the proposed training regime. The processor may be further adapted to display a visual representation of the computed probability risk factor prediction on a user interface.

According to a particular arrangement of the first aspect, there is provided a system for forming a simulation of a proposed training regime and providing a probability risk factor prediction in respect of one or more participants participating in the proposed training regime; the system comprising: a user input means adapted for accepting user input regarding: a proposed training regime; and training data regarding one or more participants of the proposed training regime; a storage means adapted for storing records regarding: the historical training data regarding one or more past training activity; historical participant data regarding one or more participants of the proposed training regime; and historical actual participant outcomes from the past training activity; and a system controller comprising a processor adapted to: receive user input regarding the proposed training regime and proposed participants; perform calculations to analyse the user input in conjunction with the historical training data and the historical participant outcomes; perform calculations to compute a probability risk factor prediction for each proposed participant as a result of participating in the proposed training regime; and display a visual representation of the computed probability risk factor prediction on a user interface.

The system may further comprise a storage means adapted for storing records. The stored records may comprise historical training data regarding one or more past training activity. The stored records may further comprise historical participant data regarding one or more participants of the proposed training regime. The stored records may further comprise historical actual participant outcomes from the past training activity.

The system may further comprise a system controller comprising a processor. The processor may be adapted to receive user input regarding the proposed training regime and proposed participants. The processor may be further adapted to perform calculations to analyse the user input in conjunction with the historical training data and the historical participant outcomes. The processor may be further adapted to perform calculations to compute a probability risk factor prediction for each proposed participant will as a result of participating in the proposed training regime. The processor may be further adapted to display a visual representation of the computed probability risk factor prediction on a user interface.

The proposed training regime comprises a proposed physical exercise plan.

The probability risk factor prediction may comprise a prediction of the risk of injury for each proposed participant as a result of participating in a proposed future training regime.

The user input regarding the proposed training regime may comprise one or more parameters selected from the group of sprint distance, jog distance, running pace, weight of one or more training aids, exercise type or similar performance metrics as would be appreciated by the skilled addressee.

The user input regarding the one or more participants may comprise one or more parameters selected from the group of age, gender, weight, height, previous injury history, athlete-reported rating of perceived exertion (RPE), athlete heart rate (HR), training location, previous historical training regime participation outcomes and other related general training and gameplay related metrics or performance and physiological related metrics commonly utilised by sports professionals. The collection of such parameters can be generalised as athlete metadata and can be specifically related to individual athletes or can be generalised to an entire sporting team as required.

In the event that one of the participants has a history of an injury, the historical training data regarding one or more participants of the proposed training regime may further comprise an injury modifier which decreases as a function of time from the time of the initial injury such that a calculated prediction of probability risk factor for the participant in a future proposed training regime is increased by the injury modifier for a predetermined time period after the initial injury.

The injury modifier may decrease exponentially with respect to time commencing from the time of the initial injury. In alternate arrangements, the injury modifier may decrease in accordance with other smoothly varying functions, for example, polynomial, logarithmic or linear functions, or alternatively still the injury modifier may decrease in a piecewise manner such as in a step function. The decrease in the injury modifier may also decrease in a unique manner depending upon the type of injury that the participant may have suffered, e.g. a haematoma/bruising, bone contusion, soft-tissue injury etc. may all require unique recovery period profiles before the participant can be expected to return to full activity without a greater risk of recurrent injury. In further arrangements, the injury modifier may comprise a custom function based on the severity and frequency of past injuries suffered by a particular participant. In further arrangements still, the decay profile of the injury modifier may comprise a sliding convolution derived directly from the actual training data as a function of the A.I. modelling. The injury modifier may decrease with respect to time commencing from the time of the initial injury in accordance with a custom time-based function based on the frequency and/or severity of past participant injuries.

The simulation of the proposed training regime may be represented by an artificial neural network adapted to accept input parameters obtained by pre-processing of raw data. The raw data may comprise the historical training and participant data retrieved from the storage means. The raw data may further comprise participant data received from the input means. The raw data may further comprise proposed training regime data received from the input means.

The pre-processing of the raw data may comprise one or more data processing procedures. The data processing procedures may comprise data smoothing. The data processing procedures may further comprise data normalisation. The data processing procedures may further comprise data aggregation. The data processing procedures may further comprise Synthetic Minority Over-sampling Technique (SMOTE). The data processing procedures may further comprise and oversample bagging.

The input to the artificial neural network may comprise an input vector comprising an aggregation of input parameters over a predetermined time period.

The artificial neural network may be adapted to simulate a predicted probability risk factor for each participant member of a team comprising a plurality of participant members, wherein the team is scheduled to participate in a future proposed training regime.

The system may further comprise means to select a predetermined time window for analysis of participant probability risk factor during a proposed training regime comprising a plurality of training exercises.

The prediction of probability risk factor may be provided in real time on selection of a predetermined time window indicative of the duration of the proposed training regime.

The prediction of probability risk factor may be provided in real time on modifications of the proposed training regime.

According to a second aspect of the invention, there is provided a system for forming a simulation of a proposed training regime and providing a probability risk factor prediction. The system may comprise one or more active artificial neural networks adapted for simulation of one or more participants of a proposed exercise training regime such that the simulation provides outputs comprising a predicted probability risk factor for one or more the participants of the proposed training regime.

According to a particular arrangement of the second aspect, there is provided a system for forming a simulation of a proposed training regime and providing a probability risk factor prediction, the system comprising one or more active artificial neural networks adapted for simulation of one or more participants of a proposed exercise training regime such that the simulation provides outputs comprising a predicted probability risk factor for one or more the participants of the proposed training regime.

The system may further comprise one or more training artificial neural networks correlated to each of the active artificial neural networks, and wherein: on receipt of data comprising data in respect of the proposed exercise training regime and/or in respect of one or more of the participants the system is adapted to perform computational training of each the training artificial neural network to provide a predicted probability risk factor; and wherein in the event the predicted probability risk factor obtained from the training artificial neural network provides a more accurate predicted probability risk factor than that obtained from a corresponding one of the active artificial neural networks, the corresponding the active artificial neural network is replaced by the training artificial neural network to form a new active artificial neural network, and a new training artificial neural network is formed for ongoing training of the artificial neural networks.

According to a third aspect of the invention, there is provided a method of planning a proposed training regime for one or more training participants. The method may comprise the step of entering user input to a system controller. The user input may comprise information regarding a proposed training regime and one or more proposed training participants. The system controller may comprise user input means for accepting the user input. The system controller may further comprise storage means adapted to store records regarding: historical training data; historical training regimes and historical actual participant outcomes from the historical training regimes. The system controller may further comprise a processor adapted to perform calculations. The processor may be adapted to analyse the user input in conjunction with the historical training data and the historical participant outcomes. The processor may be further adapted to compute a probability risk factor prediction comprising a prediction of the risk of injury for each proposed participant as a result of participating in the proposed training regime. The processor may be further adapted to receive calculated probability risk factor prediction data including prediction of the risk of injury for each proposed participant as a result of participating in the proposed training regime. The processor may be further adapted to modify the proposed training regime to minimise the risk of injury for one or more of the proposed participants.

According to a particular arrangement of the third aspect, there is provided a method of planning a proposed training regime for one or more training participants, the method comprising the steps of: entering user input to a system controller, the user input comprising information regarding a proposed training regime and one or more proposed training participants, the system controller comprising user input means for accepting the user input; storage means adapted to store records regarding: historical training data; historical training regimes and historical actual participant outcomes from the historical training regimes; a processor adapted to perform calculations to: analyse the user input in conjunction with the historical training data and the historical participant outcomes; and compute a probability risk factor prediction comprising a prediction of the risk of injury for each proposed participant as a result of participating in the proposed training regime; receiving calculated probability risk factor prediction data including prediction of the risk of injury for each proposed participant as a result of participating in the proposed training regime; and modifying the proposed training regime to minimise the risk of injury for one or more of the proposed participants.

According to a fourth aspect of the invention, there is provided readable medium storing computer-executable. When executed by a computer, computer-executable instructions may cause the computer to perform a method for planning a proposed training regime for one or more training participants. The computer may comprise a user input means; storage means; and a processor. The method may comprise the step of receiving user input comprising information regarding a proposed training regime and one or more proposed training participants. The method may further comprise the step of effecting the processor to retrieve records from a storage means, the records comprising historical training data, historical training regimes and historical actual participant outcomes from the historical training regimes. The method may further comprise the step of effecting the processor to perform calculations to analyse the user input in conjunction with the historical training data and the historical participant outcomes. The method may further comprise the step of effecting the processor to perform calculations to compute a probability risk factor prediction comprising a prediction of the risk of injury for one or more of the proposed participant as a result of participating in the proposed training regime. The method may further comprise the step of displaying a visual representation of the computed probability risk factor prediction on a user interface.

According to a particular arrangement of the fourth aspect, there is provided a computer-readable medium storing computer-executable instructions that when executed by a computer cause the computer to perform a method for planning a proposed training regime for one or more training participants, the computer comprising a user input means; storage means; and a processor; wherein the method comprises the steps of: receiving user input comprising information regarding a proposed training regime and one or more proposed training participants; effecting the processor to retrieve records from a storage means, the records comprising historical training data, historical training regimes and historical actual participant outcomes from the historical training regimes; effecting the processor to perform calculations to analyse the user input in conjunction with the historical training data and the historical participant outcomes; effecting the processor to perform calculations to compute a probability risk factor prediction comprising a prediction of the risk of injury for one or more of the proposed participant as a result of participating in the proposed training regime; and displaying a visual representation of the computed probability risk factor prediction on a user interface.

The method may further comprise the step of accepting user input with respect to the displayed visual representation, the user input comprising a selection of a time window with respect to the proposed training regime, wherein, upon receipt of the user input, the processor is effecter to perform calculations to compute a probability risk factor prediction for at least one or more participants at the end of the selected time window.

According to a fifth aspect of the invention, there is provided a computer program product having a computer readable medium having a computer program recorded therein for forming a simulation of a proposed training regime and providing a probability risk factor prediction for one or more proposed participants of the proposed training regime. The computer may comprise a user input means; storage means; and a processor. The computer program product may comprise computer program code means for receiving user input comprising information regarding a proposed training regime and one or more proposed training participants. The computer program product may further comprise computer program code means for effecting a processor to retrieve records from a storage means, the records comprising historical training data; and historical training regimes and historical actual participant outcomes from the historical training regimes. The computer program product may further comprise computer program code means for effecting a processor to perform calculations to analyse the user input in conjunction with the historical training data and the historical participant outcomes. The computer program product may further comprise computer program code means for effecting a processor to perform calculations to compute a probability risk factor prediction comprising a prediction of the risk of injury for one or more of the proposed participant as a result of participating in the proposed training regime. The computer program product may further comprise computer program code means for displaying a visual representation of the computed probability risk factor prediction on a user interface.

According to a particular arrangement of the fifth aspect, there is provided a computer program product having a computer readable medium having a computer program recorded therein for forming a simulation of a proposed training regime and providing a probability risk factor prediction for one or more proposed participants of the proposed training regime, the computer comprising a user input means; storage means; and a processor; the computer program product comprising: computer program code means for receiving user input comprising information regarding a proposed training regime and one or more proposed training participants; computer program code means for effecting a processor to retrieve records from a storage means, the records comprising historical training data; and historical training regimes and historical actual participant outcomes from the historical training regimes; computer program code means for effecting a processor to perform calculations to analyse the user input in conjunction with the historical training data and the historical participant outcomes; computer program code means for effecting a processor to perform calculations to compute a probability risk factor prediction comprising a prediction of the risk of injury for one or more of the proposed participant as a result of participating in the proposed training regime; and computer program code means for displaying a visual representation of the computed probability risk factor prediction on a user interface.

According to a sixth aspect of the invention, there is provided a computer program for forming a simulation of a proposed training regime and providing a probability risk factor prediction for one or more proposed participants of the proposed training regime. The program may comprise code for receiving user input comprising information regarding a proposed training regime and one or more proposed training participants. The program may further comprise code for effecting a processor to retrieve records from a storage means, the records comprising historical training data; and historical training regimes and historical actual participant outcomes from the historical training regimes. The program may further comprise code for effecting a processor to perform calculations to analyse the user input in conjunction with the historical training data and the historical participant outcomes. The program may further comprise code for effecting a processor to perform calculations to compute a probability risk factor prediction comprising a prediction of the risk of injury for one or more of the proposed participant as a result of participating in the proposed training regime. The program may further comprise code for displaying a visual representation of the computed probability risk factor prediction on a user interface.

According to a particular arrangement of the sixth aspect, there is provided a computer program for forming a simulation of a proposed training regime and providing a probability risk factor prediction for one or more proposed participants of the proposed training regime, the program comprising: code for receiving user input comprising information regarding a proposed training regime and one or more proposed training participants; code for effecting a processor to retrieve records from a storage means, the records comprising historical training data; and historical training regimes and historical actual participant outcomes from the historical training regimes; code for effecting a processor to perform calculations to analyse the user input in conjunction with the historical training data and the historical participant outcomes; code for effecting a processor to perform calculations to compute a probability risk factor prediction comprising a prediction of the risk of injury for one or more of the proposed participant as a result of participating in the proposed training regime; and code for displaying a visual representation of the computed probability risk factor prediction on a user interface.

According to an seventh aspect of the invention, there is provided a system comprising one or more processors. The system may further comprise memory coupled to the one or more processors and configured to store instructions. When executed by the one or more processors, the instructions may cause the processors to perform an operation comprising receiving user input comprising information regarding a proposed training regime and one or more proposed training participants. The instructions may cause the processors to perform a further operation comprising effecting a processor to retrieve records from a storage means. The records may comprise historical training data, historical training regimes and historical actual participant outcomes from the historical training regimes. The instructions may cause the processors to perform a further operation comprising effecting a processor to perform calculations to analyse the user input in conjunction with the historical training data and the historical participant outcomes. The instructions may cause the processors to perform a further operation comprising effecting a processor to perform calculations to compute a probability risk factor prediction comprising a prediction of the risk of injury for one or more of the proposed participant as a result of participating in the proposed training regime. The instructions may cause the processors to perform a further operation comprising displaying a visual representation of the computed probability risk factor prediction on a user interface.

According to a particular arrangement of the seventh aspect, there is provided a system comprising: one or more processors; memory coupled to the one or more processors and configured to store instructions, which, when executed by the one or more processors, causes the processors to perform operations comprising: receiving user input comprising information regarding a proposed training regime and one or more proposed training participants; effecting a processor to retrieve records from a storage means, the records comprising historical training data; and historical training regimes and historical actual participant outcomes from the historical training regimes; effecting a processor to perform calculations to analyse the user input in conjunction with the historical training data and the historical participant outcomes; effecting a processor to perform calculations to compute a probability risk factor prediction comprising a prediction of the risk of injury for one or more of the proposed participant as a result of participating in the proposed training regime; and displaying a visual representation of the computed probability risk factor prediction on a user interface.

According to an eighth aspect of the invention, there is provided a non-transitory storage device storing instructions. When executed by one or more processors, the one or more processors may be caused to perform an operation comprising receiving user input comprising information regarding a proposed training regime and one or more proposed training participants. The one or more processors may be caused to perform a further operation comprising effecting a processor to retrieve records from a storage means, the records comprising historical training data; and historical training regimes and historical actual participant outcomes from the historical training regimes. The one or more processors may be caused to perform a further operation comprising effecting a processor to perform calculations to analyse the user input in conjunction with the historical training data and the historical participant outcomes. The one or more processors may be caused to perform a further operation comprising effecting a processor to perform calculations to compute a probability risk factor prediction comprising a prediction of the risk of injury for one or more of the proposed participant as a result of participating in the proposed training regime. The one or more processors may be caused to perform a further operation comprising displaying a visual representation of the computed probability risk factor prediction on a user interface.

According to a particular arrangement of the eighth aspect, there is provided a non-transitory storage device storing instructions that, when executed by one or more processors, cause the one or more processors to perform operations comprising: receiving user input comprising information regarding a proposed training regime and one or more proposed training participants; effecting a processor to retrieve records from a storage means, the records comprising historical training data; and historical training regimes and historical actual participant outcomes from the historical training regimes; effecting a processor to perform calculations to analyse the user input in conjunction with the historical training data and the historical participant outcomes; effecting a processor to perform calculations to compute a probability risk factor prediction comprising a prediction of the risk of injury for one or more of the proposed participant as a result of participating in the proposed training regime; and displaying a visual representation of the computed probability risk factor prediction on a user interface.

According to a ninth aspect of the invention, there is provided a computer program product for simulating a proposed training regime and providing a probability risk factor prediction, the computer program product comprising a computer readable storage medium having program code embodied therewith. The program code may be executable by a processor to receive user input comprising information regarding a proposed training regime and one or more proposed training participants. The program code may be further executable by the processor to effect the processor to retrieve records from a storage means, the records comprising historical training data; and historical training regimes and historical actual participant outcomes from the historical training regimes. The program code may be further executable by the processor to train statistical models in respect of simulating the training regime and each of the participants of the proposed training regime. The training of the statistical models may comprise effecting a processor to perform calculations to analyse the user input in conjunction with the historical training data and the historical participant outcomes. The training of the statistical models may further comprise effecting a processor to perform calculations to compute a probability risk factor prediction comprising a prediction of the risk of injury for one or more of the proposed participant as a result of participating in the proposed training regime;

The program code may be further executable by the processor to compute a probability risk factor prediction with respect to at least one or more of the participants. The program code may be further executable by the processor to displaying a visual representation of each the computed probability risk factor prediction on a user interface.

According to a particular arrangement of the ninth aspect, there is provided a computer program product for simulating a proposed training regime and providing a probability risk factor prediction, the computer program product comprising a computer readable storage medium having program code embodied therewith, the program code executable by a processor to: receiving user input comprising information regarding a proposed training regime and one or more proposed training participants; effecting a processor to retrieve records from a storage means, the records comprising historical training data; and historical training regimes and historical actual participant outcomes from the historical training regimes; train statistical models in respect of simulating the training regime and each of the participants of the proposed training regime, the training of the statistical models comprising: effecting a processor to perform calculations to analyse the user input in conjunction with the historical training data and the historical participant outcomes; and effecting a processor to perform calculations to compute a probability risk factor prediction comprising a prediction of the risk of injury for one or more of the proposed participant as a result of participating in the proposed training regime; computing a probability risk factor prediction with respect to at least one or more of the participants; and displaying a visual representation of each the computed probability risk factor prediction on a user interface.

### BRIEF DESCRIPTION OF THE DRAWINGS

Further features of the present invention are more fully described in the following description of several non-limiting embodiments/arrangements thereof. This description is included solely for the purposes of exemplifying the present invention. It should not be understood as a restriction on the broad summary, disclosure or description of the invention as set out above. The description will be made with reference to the accompanying drawings in which:

FIG. 1 shows a computing device adapted for simulation of a proposed training regime and providing a probability risk factor prediction in respect of one or more participants participating in a proposed training regime;

FIG. 2 shows a visual representation of a user interface according to a particular arrangement of the system of FIG. 1 disclosed herein;

FIG. 3 shows an example method of planning a proposed training regime according to the systems and methods disclosed herein;

FIG. 4 shows a graphical overview representation of a particular example arrangement of the system of FIG. 1;

FIG. 5 shows a representation of the data flow for the AI Model Training Server (AMTS);

FIG. 6 shows a representation of the training process for the AI model training;

FIG. 7 shows a representation of the raw data pre-processing procedure as disclosed herein;

FIG. 8 shows a representation of an array of processed data according to the procedure of FIG. 7;

FIGS. 9A and 9B show a representation of the entity relation and interaction with the data store in the present sports injury prediction model;

FIG. 10 shows a representation of the input and output variable to the artificial neural networks of the example arrangement as disclosed herein;

FIG. 11 shows a representation of the model parameter selection;

FIG. 12 shows an example output graph of the comparison of validation data from the present example arrangement of injuries vs predicted outcome; and

FIG. 13 shows an example computing device computing device on which the various embodiments described herein may be implemented in accordance with arrangements or embodiments of the present invention.

### DEFINITIONS

The following definitions are provided as general definitions and should in no way limit the scope of the present invention to those terms alone, but are put forth for a better understanding of the following description.

Unless defined otherwise, all technical and scientific terms used herein have the same meaning as commonly understood by those of ordinary skill in the art to which the invention belongs. It will be further understood that terms used herein should be interpreted as having a meaning that is consistent with their meaning in the context of this specification and the relevant art and will not be interpreted in an idealized or overly formal sense unless expressly so defined herein. For the purposes of the present invention, additional terms are defined below. Furthermore, all definitions, as defined and used herein, should be understood to control over dictionary definitions, definitions in documents incorporated by reference, and/or ordinary meanings of the defined terms unless there is doubt as to the meaning of a particular term, in which case the common dictionary definition and/or common usage of the term will prevail.

For the purposes of the present invention, the following terms are defined below.

The articles “a” and “an” are used herein to refer to one or to more than one (i.e. to at least one) of the grammatical object of the article. By way of example, “an element” refers to one element or more than one element.

The term “about” is used herein to refer to quantities that vary by as much as 30%, preferably by as much as 20%, and more preferably by as much as 10% to a reference quantity. The use of the word ‘about’ to qualify a number is merely an express indication that the number is not to be construed as a precise value.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

Any one of the terms: “including” or “which includes” or “that includes” as used herein is also an open term that also means including at least the elements/features that follow the term, but not excluding others. Thus, “including” is synonymous with and means “comprising”.

The term, “real-time”, for example “displaying real-time data”, refers to the display of the data without intentional delay, given the processing limitations of the system and the time required to accurately measure the data.

The term. “near-real-time”, for example “obtaining real-time or near-real-time data” refers to the obtaining of data either without intentional delay (“real-time”) or as close to real-time as practically possible (i.e. with a small, but minimal, amount of delay whether intentional or not within the constraints and processing limitations of the of the system for obtaining and recording or transmitting the data).

Although any methods and materials similar or equivalent to those described herein can be used in the practice or testing of the present invention, preferred methods and materials are described. It will be appreciated that the methods, apparatus and systems described herein may be implemented in a variety of ways and for a variety of purposes. The description here is by way of example only.

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

“Computer-readable media/medium”, as used herein, refers to media or a medium that stores signals, instructions and/or data. A computer-readable medium may take forms, including, but not limited to, non-volatile media, and volatile media. Non-volatile media may include, for example, optical disks, magnetic disks, and so on. Volatile media may include, for example, semiconductor memories, dynamic memory, and so on. Common forms of a computer-readable medium may include, but are not limited to, a floppy disk, a flexible disk, a hard disk, a magnetic tape, other magnetic medium, an ASIC, a CD, other optical medium, a RAM, a ROM, a memory chip or card, a memory stick, and other media from which a computer, a processor or other electronic device can read.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the summary above and the description below, all transitional phrases such as “comprising,” “including,” “carrying,” “having,” “containing,” “involving,” “holding,” “composed of,” and the like are to be understood to be open-ended, i.e., to mean “including but not limited to”. Only the transitional phrases “consisting of” and “consisting essentially of” alone shall be closed or semi-closed transitional phrases, respectively.

For the purpose of this specification, where method steps are described in sequence, the sequence does not necessarily mean that the steps are to be carried out in chronological order in that sequence, unless there is no other logical manner of interpreting the sequence.

In addition, where features or aspects of the invention are described in terms of Markush groups, those skilled in the art will recognise that the invention is also thereby described in terms of any individual member or subgroup of members of the Markush group.

### DETAILED DESCRIPTION

It should be noted in the following description that like or the same reference numerals in different embodiments denote the same or similar features.

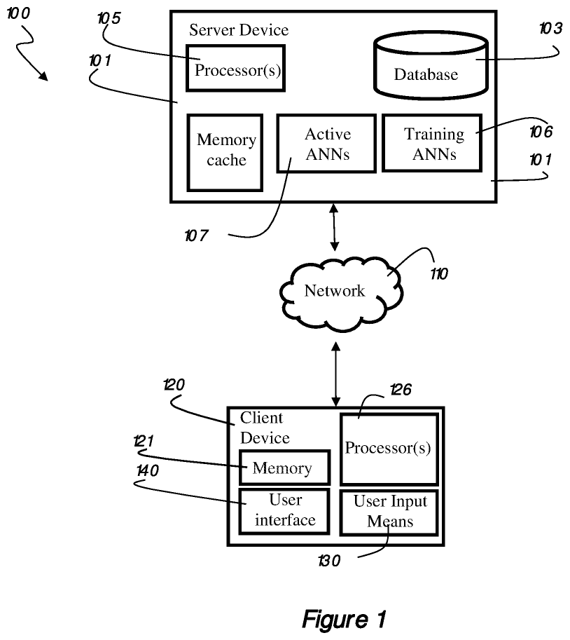

FIG. 1 shows a system 100 of computing devices adapted for simulation of a proposed training regime and providing a probability risk factor prediction in respect of one or more participants participating in a proposed training regime in a program application adapted for use on a client/server computing device arrangement. In particular arrangements, system 100 comprises a web-based application for professional sports teams and individuals to manage will to manage training regimes and minimise injuries to their players.

As will be described in further detail below, the system 100 provides a system for simulation of a proposed training regime and providing a probability risk factor prediction in respect of one or more participants participating in the proposed training regime.

The system 100 comprises a user input means 130 in client computing device 120 adapted for accepting user input. The user input comprises data regarding a proposed training regime. The user input further comprises data regarding training data regarding one or more participants of the proposed training regime.

The client device 120 is connected to a system controller comprising a server device 101 via a network 110. Network 110 may include, for example Ethernet, Bluetooth or IEEE 802.X wireless networks and may, for example, be the internet.

The server device 101 further comprises a storage means 103 comprising a database adapted for storing records. The stored records may comprise historical training data regarding one or more past training activity. The stored records may further comprise historical participant data regarding one or more participants of the proposed training regime. The stored records may further comprise historical actual participant outcomes from the past training activity.

The server device 101 further comprises a processor 105. The processor is adapted to receive user input from client device 120 regarding the proposed training regime and proposed participants of the proposed training regime which, in embodiments and arrangements disclosed herein comprises a physical exercise training program for a sporting team. The processor 105 is further adapted to perform calculations to analyse the user input in conjunction with the historical training data and historical participant outcomes. The processor 105 is further adapted to perform calculations to compute a probability risk factor prediction for each proposed participant will as a result of participating in proposed training regime. The processor may be further adapted to display a visual representation of the computed probability risk factor prediction on a user interface 140 of client device 120.

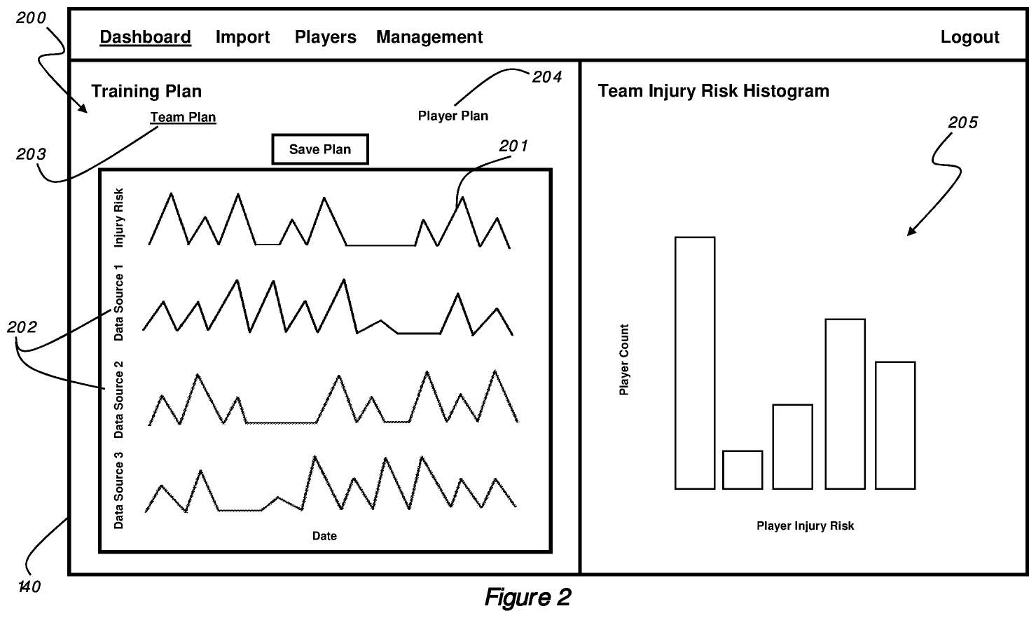

In particular arrangements, the user interface 140 may comprise a webpage adapted to display the information generated by the server device to the end user. End users can update and modify this information as deemed necessary. The user interface 140 also provides a login portal for users to connect to the server device; whereupon when connected, the user is presented with a graphical representation will of a current iteration of a proposed training regime (should one exist in the database storage 103 of server device 101). The user is also presented with a visual representation of an overview of the team member participants and predicted injury risk factor calculations for each participant of the team, where the injury risk factor calculation results are expressed as a probability of a team participant being injured as a result of the planned programme. An example of such visual representation 200 on user interface 140 is shown in FIG. 2.

In the event that a training regime is not already present in the storage database 103 of server 101, then the user proceeds to import and upload to server 101 historical sports training data which is collected by the server 101 and aggregated to form a prediction model for future training regimes. In particular arrangements particularly with respect to trainings and relations for sports related events, specific signals are collected by server 101, aggregated and analysed including, but not limited to: Training Load Information including sprint distance, jog distance, etc.; Athlete Metadata including age, gender, weight, height, and previous injury history. Previous injury history for athletes is preferably correlated against historical training session data so as to be used to assist in predicting the probability of athlete injuries in future training sessions.

Once the historical training and participant data has been collected and aggregated by server 101, the server 101 is directed to commence an artificial intelligence process to map the raw input training and participant data to sport-specific outcomes including injury and non-injury. This is done by utilizing an artificial neural network (ANN) training model 106 to predict the probability risk factors for participant injury as a result of participating in the training regime. Clustering of multiple similar datasets can optionally be used to increase the pool size of raw data for the training of the training ANN model 106. For example, raw data relating to training regimes and/or athlete participants across different sporting teams, sports, individuals and/or athlete positions within a particular team may be clustered together for improved training of the training ANN model 106. The artificial neural network is preferably adapted to simulate a predicted probability risk factor for each participant member of a team comprising a plurality of participant members, wherein the team is scheduled to participate in a future proposed training regime.

Further optionally, additional data sampling and analysis techniques may be employed to improve the quality of data used to train the training ANN model 106 for example Synthetic Minority Over-sampling Technique (SMOTE).

Once the training ANN model 106 is deemed to provide a statistically significant prediction of the injury risk for participants participating in a future training regime, the training ANN model 106 designated as an active ANN model 107. Active ANN model 107 is then used by processor 105 of server 101 for calculation of the probably risk factors for the training regime. As new data is continually added to the server database 103 regarding will training regimes, participant information updates will and actual recorded injuries as a result of training sessions, the training ANN model 106 is continually updated with will the new raw data to improve the predictive capability of the training ANN model will 106. The predictive performance of the training ANN model 106 is continually monitored once it by server 101 and when the quality of the injury risk predictions generated by the training ANN model 106 exceeds that of the current active ANN model 107, then the active ANN model 107 is replaced by the superior training ANN model 106 and used for ongoing predictions.

The probability risk factor prediction comprises a prediction of the risk of injury for each proposed participant as a result of participating in a proposed future training regime.

The user input regarding the proposed training regime comprises one or more parameters selected from the group of sprint distance, jog distance, running pace, heart rate metrics, player biometric measures, player effort intensity measures, weight of one or more training aids, exercise type. The user input regarding the one or more participants comprises one or more parameters selected from the group of age, gender, weight, height, previous injury history, previous historical training regime participation outcomes.

In the event that one of the participants has a history of an injury, the historical training data regarding one or more participants of the proposed training regime may further comprise an injury modifier 803 which will affect their predicted injury risk calculations. The injury risk modifier is an input to the injury risk calculation which decreases as a function of time from the time of the initial injury such that a calculated prediction of probability risk factor for the participant in a future proposed training regime is increased by the injury modifier for a predetermined time period after the initial injury. The injury modifier is adapted so as to decrease with respect to time commencing from the time of the initial injury to reflect the participant's recovery from the injury, until such point as the athlete is considered to be fully recovered from injury and the injury modifier is consequently reduced to zero. Multiple injuries in succession increase the injury risk factor by a configurable amount which is optimized in the training regime. In most arrangements, the injury risk modifier is adapted to decrease exponentially with respect to time, this is to mimic the real-world influence of past injury on future injury instances, and it is applied as such to assist the AI in learning the influence of previous injury. In particular arrangements comprising sparse data sets with limited historical injury data, the injury risk modifier may simply be implemented as just a simple binary indication of the occurrence of a prior injury rather than a function of decreasing influence, which can provide improved prediction outcomes where such historical injury data is limited.

The simulation of the proposed training regime may be represented by an artificial neural network (ANN) adapted to accept input parameters obtained by pre-processing of raw data. The raw data may comprise the historical training and participant data retrieved from the storage means. The raw data may further comprise participant data received from the input means. The raw data may further comprise proposed training regime data received from the input means.

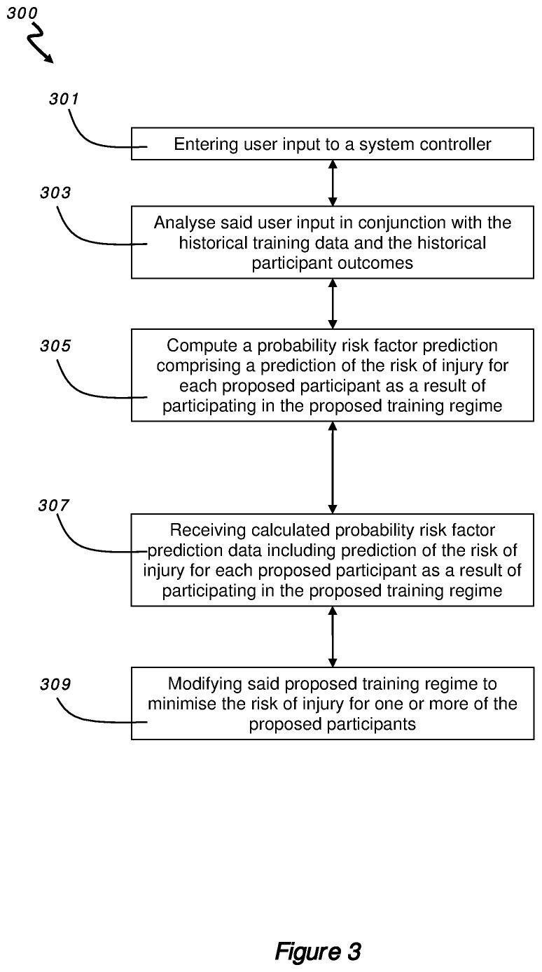

Using the training regime prediction model disclosed herein uses of the model, in particular team trainers and their assistants, may utilise the system to prepare and modify a training regime for a team or individual athletes within such team by a method 300 planning a proposed training regime for one or more training participants as shown in FIG. 3 as follows: method of planning a proposed training regime for one or more training participants, the method comprising the steps of: entering user input to a system controller 301, the user input comprising, the user information regarding a proposed training regime and one or more proposed training participants, the system controller comprising user input means for accepting the user input; storage means adapted to store records regarding: historical training data; historical training regimes and historical actual participant outcomes from the historical training regimes; and a processor adapted to perform calculations to: analyse 303 the user input in conjunction with the historical training data and the historical participant outcomes; and compute 305 a probability risk factor prediction comprising a prediction of the risk of injury for each proposed participant as a result of participating in the proposed training regime; receiving 307 calculated probability risk factor prediction data including prediction of the risk of injury for each proposed participant as a result of participating in the proposed training regime; and modifying 309 the proposed training regime to minimise the risk of injury for one or more of the proposed participants.

### Front-End Architecture

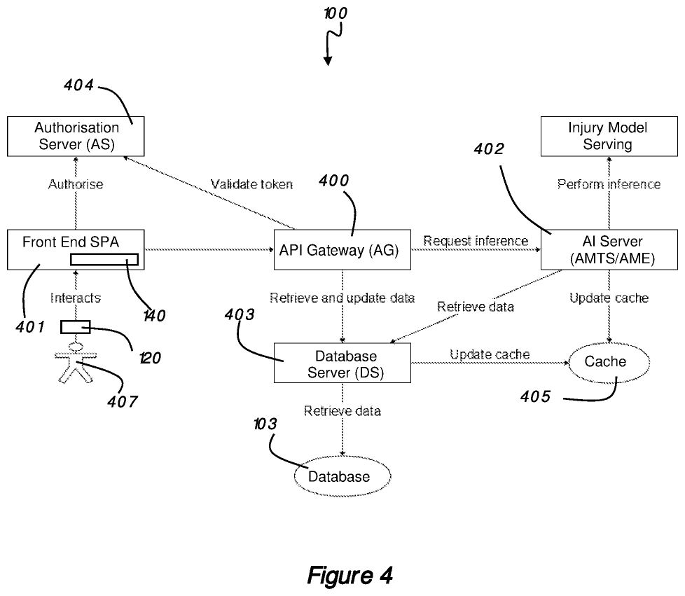

FIG. 4 shows a graphical overview representation of a particular example arrangement of system 100. Where specific software packages or routines are mentioned in the following, it will be appreciated that such mentioned packages are included herein merely as an example of one possible package which may be used in conjunction with the systems & methods disclosed herein. Such mentioned packages are not intended to be exclusively required in the systems and methods. Other software packages and/or routines providing equivalent functionality to those disclosed herein will be readily appreciated by the skilled addressee for performing an equivalent function and accordingly may be readily interchanged with packages and/or routines disclosed herein whilst remaining within the spirit of the invention disclosed herein.

The frontend architecture, Application Server (AS), is comprised of two key elements. In this particular arrangement, an API Gateway (AG) 400 and a Single Page Web Application (SPA) 401 of FIG. 4. The SPA 401 implements the User Interface 140. The AG 400 is implemented via a .NET application server and proxies requests from the SPA 401 to the relevant services. A user 407 with client device 120 interacts directly with the SPA 401 via user interface 140.

In the present example arrangement, the backbone of the AS is that of a web server such as, for example, a Kestrel server. This web server comprises of a web server instance for hosting the web site accessible by the client device 120, as well as the API Gateway controls that channel requests to the relevant services in the Backend.

The Application Server can be hosted in most Cloud Environments (AWS/Azure). The Application Server responds to both input from the front-end web app; to serve response to client input in terms of ANN processed responses, as well as initiate ANN training regimes when necessary. The AG 400 communicates with Database Server (DS) 403 to push and pull results from front-end posts and queries. It also is responsible for channeling requests to the AI Model Training Server (AMTS)/AI Model Evaluation (AME) 402 when required.

The SPA application is, for example, an ASP.NET MVC application that consists of a Bundled Angular 4 Client-Side Application (to provide a client-side interactive & responsive environment for the web application—as would be appreciated by the skilled addressee, Angular utilises Javascript to perform logic to manipulate server responses and HTML elements without successive queries to the server). To support smoother operation Angular has been added to the MVC project to allow pages with multiple views to be traversed as a Single Page Application (SPA) in style. This allows greater UI feel and cases data fetch computational loading. As would be appreciated by the skilled addressee, Angular is a client side MVC framework maintained by google that implements data-binding as well as, dependency injection (DI) and supports routing (enabling SPA architectures). It is used to provide data binding between the data pulled and the GUI for the User.

The SPA commands data from the Database Server 403 (via the AG) and binds these to the data models (specified within the Database Server and SPA itself) as requested from the user (through the SPA). The SPA modifies the view in place in response to the Client commands. This allows data manipulation/fetch and more complex visualization without having to refresh the users' browser.

The data models used to bind data are a combination of those in the Database Server and those that are application specific and are described in FIG. 9A. The Database Server Models of the present example are described in further detail in the Backend Architecture section.

The web application in the present example arrangement has access to the Database Server and as such on server initialization, the Database Server is initialized.

Authentication to the web application is handled by an external Authentication server (AS) 404. All views and the Proxy Server are restricted by the Web Server unless authenticated with the Authentication Server. If a user is not Authenticated they are redirected to Login/Registration Page which is managed by the Authentication Server. The Authentication protocol utilized is Open ID Connect.

The application currently has 4 views, based upon the Angular4 framework. These include the predictionHQ 200, Data Import View, Player Metadata and Management.

A predictionHQ 200 View is the backbone of the application and hosts the main landing page for the application post login. It is authorized such that it can only be viewed after successful login, otherwise the user will defer to the login page. The predictionHQ is responsible for displaying Injury Risk 201 and will fetch Players, Player data 202, Post Player data and ask for Predictions. These are all contained within the relevant methods and are called from within the Angular stack towards the Proxy Server. The predictionHQ is made up of 3 components, the Team View 203, the Player View 204 and the Player Histogram 205.

A Data Import View is responsible for allowing a user to import their Team/Player data into the database 103 on server 101 via the Database Server 403. It has access to the Database Server, however the available teams attribute dictates what team data is allowed to be fetched. Within the Proxy Server is a function to handle when the user has imported a CSV/XLSX spreadsheet of data to the tool. Content preferably should be below 20 Mb and be in CSV/XLSX format otherwise an error message is displayed. Injuries can be imported via CSV/XLSX, or alternatively single HTML form upload whereby all relevant injury metadata is entered. If there is an error thrown a relevant response message will be displayed indicating to the user where the fault occurred. If an unknown error occurs, a generic error message is displayed.

The Player Metadata View is made up a simple table that consists of all the players relevant to the signed in User. It provides functionality to modify Player Metadata.

The Management View is only available to Server managers and consists of means to add/modify/remove Sports/Teams/Players and AI models from the system. It is restricted by the Authorisation Server 404.

### Back-End Architecture

The back-end architecture of the present example arrangement is represented by all components of FIG. 4 with exception to the Front End SPA 401 and API Gateway (AG) 400. The purpose of the backend is to store user and historical data sources, and train and serve AI (Neural Network) based models. The backend is queried from the front-end web application for relevant view information.

The backend architecture of the present arrangement is comprised of two key elements: The Database Server 103 and the AI Engine 402.

The Database Server 101 for the entire application employs a noSQL Database 103 and cache 405 for storage of data documents, metadata along with a file management storage service to store the raw ANN model data as pre-processed information in binary format. In this way the cache and noSQL Database 130 is used for high latency data, where query time is of importance, and persistent datastore is used for larger scale data which is less time critical. This is all routed and controlled via a .NET application server that schedules ANN training and serves the Web Application. The ANN training can be manually scheduled and co-ordinated via active management which can call functions from the Application Server (AS).

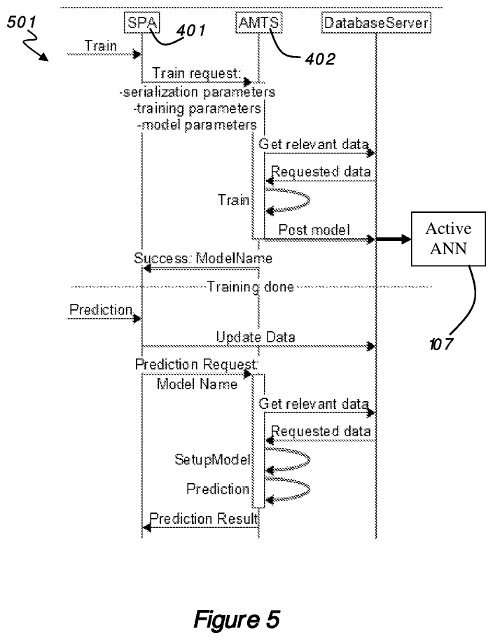

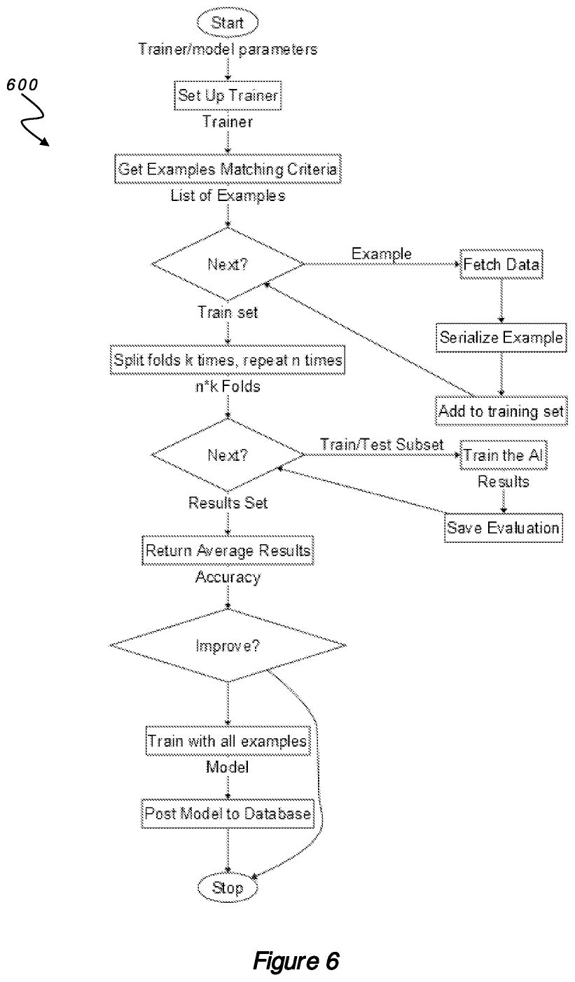

The AMTS 402 are a set of high performance computing nodes comprised of CPU and GPU clusters. The AI Training and Model Creation Program (ATMCP) is the heart of the model creation framework. The training process is shown in FIGS. 5 and 6. It runs through a Training routine 501 to train a model to a certain accuracy, then posts this model to the Database Server as active ANN model 107. The training parameters determine how the training process 600 (of FIG. 6) is executed e.g. the size of the neural network, the training mechanism, the stopping procedure and the amount of data samples used for training. The training parameters are sent to the AMTS 402 within the train request. The training parameters are stored with the model so that the model can be re-initialized at a later date with the same model and serialization configurations.

Training Examples are queried from the data by making a call to the appropriate method, and passing in the necessary arguments such as team, date and other matching example metadata. These examples are unprocessed and contain just metadata, and just serves as placeholders for creation of training examples for the AI training regime.

Specifically, the Training Examples returned contains information required for training, such as: Labels (an enum that indicates the type of event; Soft Tissue Injury, None etc.); Metadata (a Dictionary containing the metadata related to specifically identify the example; e.g. Date, Player).

As noted above, the formulation of the input data and pre-processing of this data is important for ensuring the usefulness and accuracy of the output predictions of the model. Important factors to consider in preparation of the data inputs to the model include:
The structure of the data:
   How often to sample the data? i.e. Daily as opposed to weekly or monthly;
   Which variables to measure? (This is analogous to choosing whether to take a picture or record sound of a car to distinguish if it is a car or not).
Normalization of the data:
   How each datapoint is modified to fit in the range [0,1] e.g.: z-score, min-max or box-cox normalization
Transformation of data:
   How each set of data is modified to accentuate features in the sample: e.g.: Smoothing, aggregation, rolling window transforms
Balancing of the data
   Which of the relevant set of data should be used: e.g.: UnderSampling or SMOTE

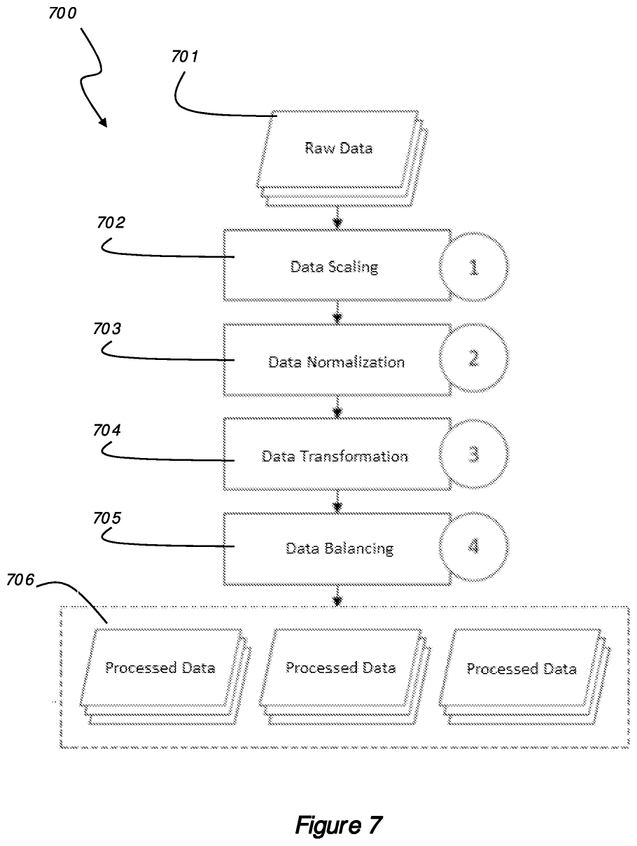

Once the examples have been queried, serialization of the examples is undertaken. In order to serialize the examples, the AMTS 402 uses the data in the training request to fetch the relevant data sources from the Database Server as Raw Data 701 as shown in FIG. 7.

Once queried, the raw data 701 is then pre-processed for the neural network by a pre-processing procedure 700 similar to that as shown in FIG. 7, note that some procedures could be omitted, combined or rearranged at will. This data pre-processing procedure includes common data processing techniques such as: Scaling 702, Normalization 703, Transformation 704 and Balancing 705. The processed data 706 is now suitable for training of the ANN training model 106.

The data is Scaled 702 linearly whereby the maximum historical value in a certain window (2 years typically) for that data source will result in a value of 1. Outliers are ignored. Other data is scaled within static ranges.

Data input to the ANN Training is first normalized 703 into the bounds [0,1], such that the initial activation function (sigmoid) is utilised and not saturated (this assists in backpropagation gradient descent). Each input in the aggregation is scaled as such. Another method used for Normalization (z-score standardization) is scaling the data to have mean 0 and a variance of one. The required method is specified in the ANN model Metadata.

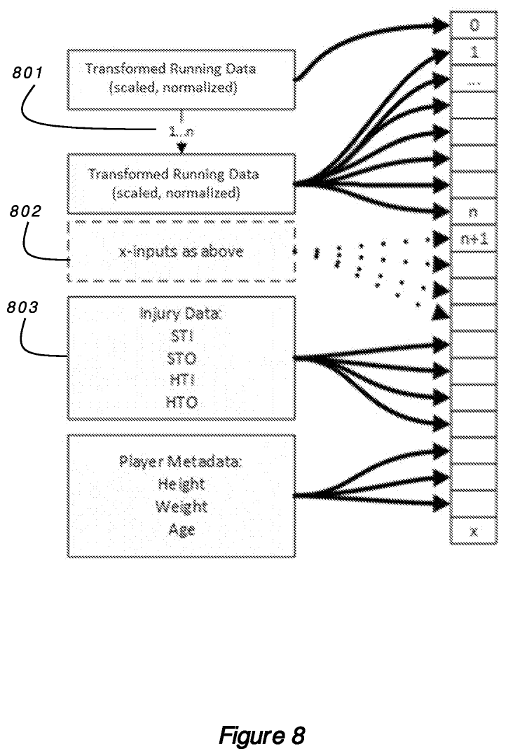

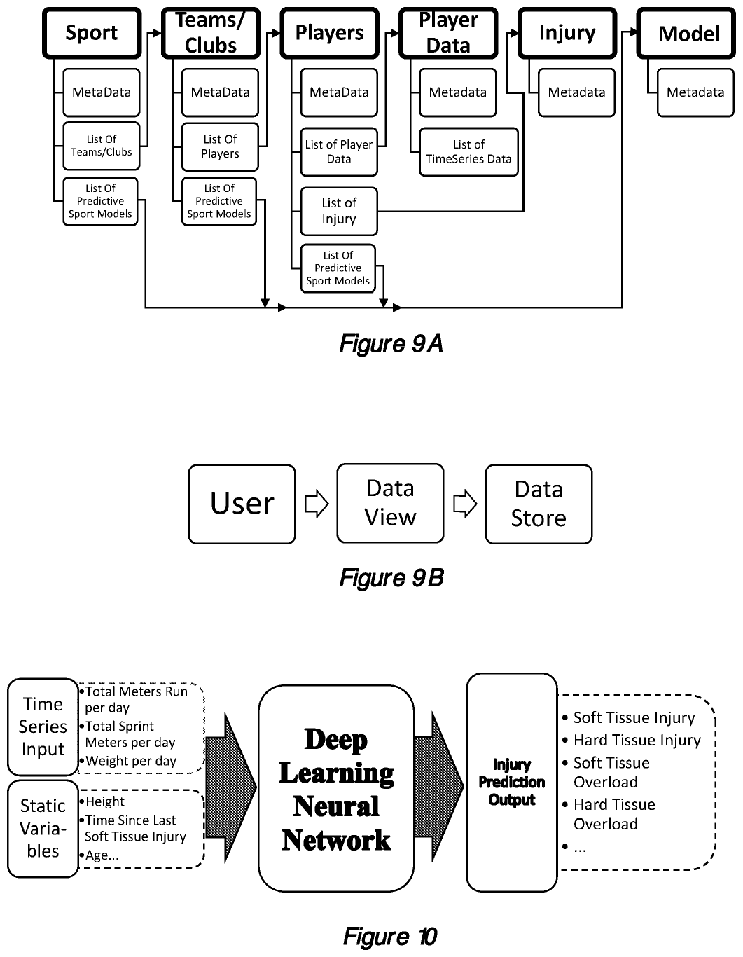

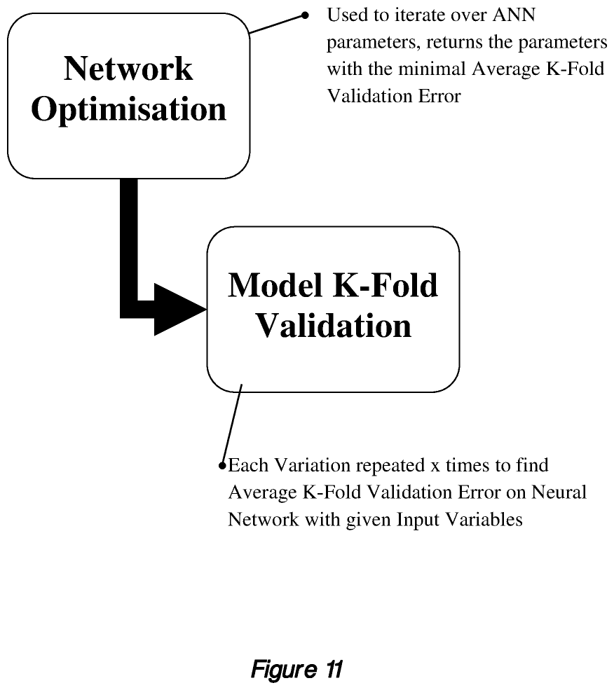

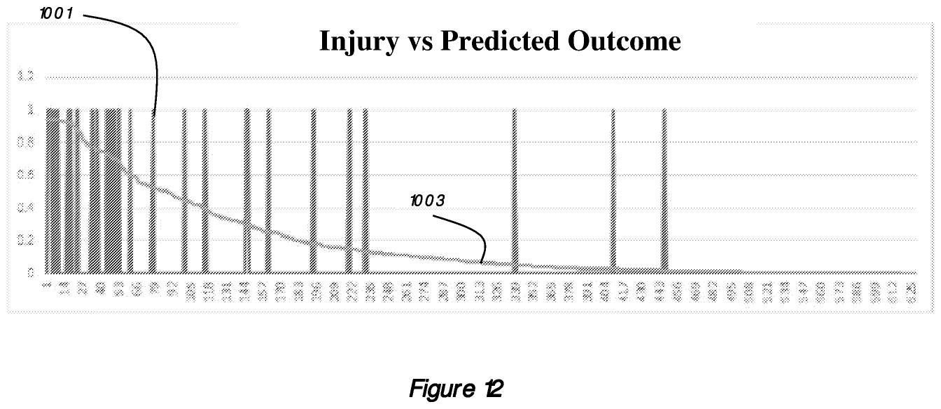

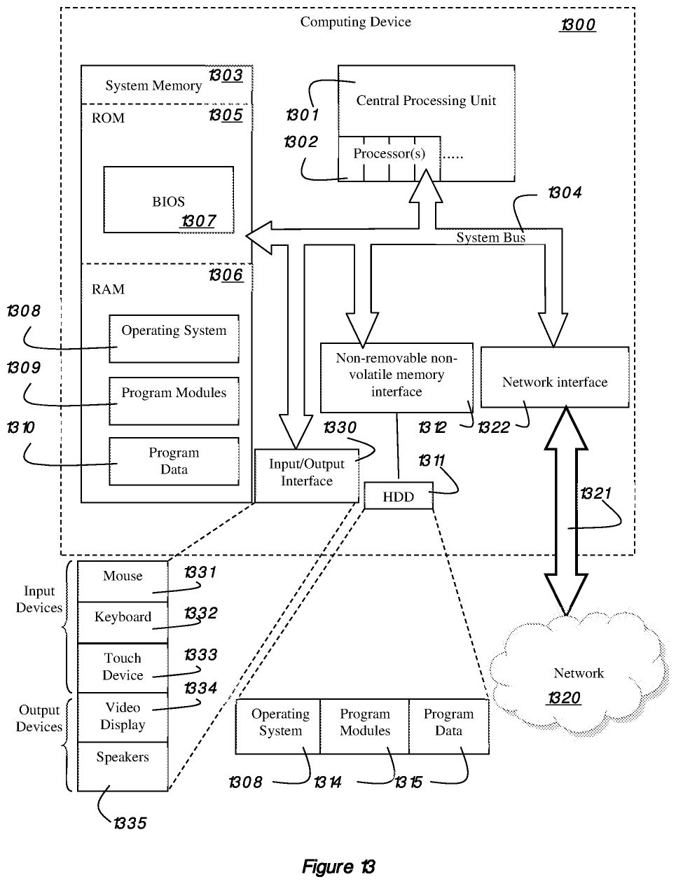

Once data has completed the Normalization procedure it is Transformed 704 into a single double array. The resulting array after transformation is of length n 801 (depending on transformation parameters) and there may be x 802 data sources 202 to be transformed. For each data source the Transformed Data is injected into the final processed data array. Any relevant injury history data for the player pertaining to the event (this is normalized days since last event) is also injected. Finally, Player metadata is included in the array as shown in FIG. 8.

A Synthetic Minority Oversampling Technique (SMOTE) is advantageously utilised to balance the heavily unbalanced data set. In the present example arrangement it is typical to see a ˜1:99 ratio of injury class events to non-Injury Class events. As such; the data must be balanced prior to training such that the larger class is not over-predicted, this is achieved via the algorithm SMOTE. SMOTE involves finding the nearest neighbours for each class in a pool, it then marries these together with the sample to produce a new sample that is similar to the original. The implementation of SMOTE used in the arrangements of the systems and methods disclosed herein is the generally accepted approach of the SMOTE algorithm, where the entire example array post transformation is utilized in creation of synthetic injury instances.

Data Balancing 705 is performed in an attempt to reduce the effects of the heavily unbalanced data set. In the present example arrangement, it is typical to see a ˜1:99 ratio of injury class events to non-Injury Class events. As such; the data must be balanced prior to training such that the larger class is not over-predicted, this is achieved via the Synthetic Minority Oversampling Technique (SMOTE) algorithm in combination with under sampling the majority class. SMOTE involves finding the nearest neighbours for each class in a pool, it then marries these together with the sample to produce a new sample that is similar to the original.

In the current example arrangement of system 100 specifically directed to modelling of injury risk prediction as result of a proposed training regime, the artificial intelligence model used is preferably a flat deep neural network. In a particular arrangement example, the AI model configuration has the following characteristics: Input nodes—Variable depending on input length; Hidden layers—2; Nodes per hidden layer—[20,15]; and Output nodes—2. Each hidden layer has dropout applied. With these parameters, the model is configured by making set up calls to the AI library used. This is where the actual Prediction Engine servers are used.

The models are trained using repeated K-Fold Validation. The data is split into k sets. The model is trained on all but one of the split data sets, and tested on the remaining. The idea being that if the evaluation on the remaining subset is good then it is likely that the model has not overfit to the training set. The model is trained until an error threshold is reached, epoch count is reached. This is repeated so that all sets are tested upon. This is then again repeated so that the accumulated results are likely to represent performance on unforeseen data.

Once the model has been trained within the k-fold evaluation above, it is evaluated on subset remaining split data. The evaluation is done by performing feed-forward operation on the model with the evaluation data, and recording the expected output and actual output from the model. In the present example the highest output is taken as the predicted value from the model. If the model supersedes performance of the current trained model it is pushed to the database, otherwise the process is repeated. The accuracy of the model is specified via many means, namely F1 score, ROC Area Under Curve and PR Area Under Curve. The ROC Area Under Curve is the primary means of determining accuracy as it caters for varying threshold levels.

Once the model is determined to be fit for saving it will be pushed to the database as active ANN 107 described above. The model may be made up of 1→N Trained ANN models 106 called an ensemble. The model(s) are serialized into a compressed format and pushed to the Datastore for later retrieval when needed with metadata such as: Trained Date (the date the model is trained); Model Name (the name of the model); Model Properties (this may include F-measure, PR AUC, ROC AUC); Fitness (a measure of the accuracy of the model, currently the ROC AUC); and Smoothing (amount of smoothing applied in the input data).

### Injury Modelling

The Sport Injury Prediction system disclosed herein consists of the three individual components; the Datastore, the AI Engine and the Web Application. Each of these components interact to provide a product to the end user however, each are individually addressable as an entity.

The Datastore (database incorporating a file management system) utilises a cloud based non-relational data store to store objects as JSON documents, as well as a file management storage service for storage of larger items as discussed in the Backend Architecture. These objects consist of: Sport, Team/Club, Player, Player Data, Injury, Prediction Model objects. These relate to each other such that each sport has its own prediction model and clubs and metadata; each club has its players, model and metadata; and each Player has its model, Player Data's, Injuries and metadata. The information may be loaded to the cloud as required via the Data Import view, and is hash encrypted prior to store and decrypted upon retrieval. FIGS. 9A and 9B describes the entity relation and interaction with the data store in the present sports injury prediction model. Each user only sees the data within their Data View. A query for all players as such will return only those players which are allowed to be accessed by that user. This is consistent for all entity objects. The AI server is a special user which has access to all data from the datastore; this allows aggregation of data across users without any user having access to other user's data.

The Prediction Engine utilises the data presented in the data store to create a prediction on the likelihood of injury. The Prediction Engine takes the raw data from the data store and performs two functions to provide data output to the data view. The Prediction Engine will first train on known data within the data store to provide a Model 106. This model is then used to create predictions 107.

In order to train; the Prediction Engine first pre-process' the data from the Data Store by retrieving and normalising the relevant fields as inputs as described in the Backend Architecture. This is typically 28-day window of a number of data sources; such as Distance Run, Sprint Meters Run, as well as static variables; height, weight, time since last injury. Each of these inputs are team-dependent and so is variant as to the amount of inputs available to the predictive model. FIG. 10 shows an example representation of the input and output variable to the artificial neural networks of the present example arrangement, while FIG. 11 is a representation of the model parameter selection in particular arrangements of the system.

Back propagation is used to configure the optimal network. The Network consists of multiple layers of a number of simulated Neurons. The model may consist of Deep Neural Network (DNN), Recurrent Neural Network (RNN) as well as Convolutional Neural Network (CNN) layers, depending on the accuracy of the specific model, the typical model, however is made up of dense layers in combination with dropout. Each layer may consist of sigmoid, tan, or any other activation function base neurons with some bias applied. Back propagation is an optimisation technique used to configure the optimal network. Gradient Decent optimization may be used to update model weights, as is standard for Supervised Learning C/R/DNNs. The cost function minimized under gradient descent is weighted linearly such that the predictions of the majority class are weighted by the ratio of majority to minority classes in the epoch. Other optimisation techniques can be used as necessary as would be appreciated by the skilled addressee. Training is stopped when model accuracy is no longer improving, or the maximum number of epochs has expired. Regularization/Dropout is performed to reduce the amount of overfitting, when training the ANN.

When users would like to use this model to make some injury prediction; the model is “fed forward”. This processing is taken place server-side to the user rather than locally on the user machine. The PredictionHQ View within the web application presents the data inputs to the users and the current prediction. The SPA Application queries the Proxy Server which will query both the data store and, if necessary, the predictive engine to display results to the user. The Individual Player View 204 has a Line graph of the time series data inputs. These data points can be modified in real time to observe impacts to the Injury Prediction. Similarly, users can view an Overall Team Plan 203 which presents a histogram of current prediction for a given team.

The Web Application is what is utilized to input the training plan for any individual. In this manner, planning out future loads allows the known loads and future loads to be concatenated and provided to the AI as if it were a known example as in FIG. 8. As an example, a trainer may have planned out 4 days into the future of training loads. A model that utilizes 28 days of historic data would therefore use 24 days of known historic loads plus the 4 planned loads to predict injury risk on the final day of their plan. On the second last day of their 4-day plan, 25 days of known loads and the 3 earlier days of the plan are used as inputs etc.

### System Performance and Results

Model Parameter Training: First using K-Fold validation, model parameters are selected including: normalization parameters, model size (hidden layers, nodes per layer), number of samples, epochs, SMOTE percentages etc. until good results are achieved, some of these parameters are selected via brute force evaluation/optimization. Once appropriate parameters are found, the model is trained utilizing all of the data, and a model is generated for use 107.

FIG. 12 shows an example output graph of the comparison of validation data from the present example arrangement of injuries vs predicted outcome. Along the x axis is a sample of a random training days pulled from a dataset: random players on random days. Included also are injuries that were reported. The model had never seen this data before.

The dark line 1001 indicates whether there was an injury. The lighter line 1003 indicates the predicted chance of injury for that particular day. Ideally, ALL the dark spikes 1001 would be seen to the left of the graph, when the model had predicted the highest chance of injury. However, variation in injury definitions and recordings lead to misclassified injury predictions. Statistics such as F1 score, PR AUC, ROC AUC are created from the training process. Currently model effectiveness is measured by ROC AUC.

The methods of simulating of a proposed training regime and providing a probability risk factor prediction and method of planning a proposed training regime for one or more training participants to minimise a risk of injury to the participants as depicted in FIG. 3 (and associated sub methods (e.g. method(s) 600 and 700 depicted in FIGS. 6 and 7 may be implemented using a computing device/computer system 1300, such as that shown in FIG. 13 wherein the processes disclosed herein may be implemented as software, such as one or more application programs executable within the computing device 1300. In particular, the steps of the methods disclosed herein are effected by instructions in the software that are carried out within the computer system 1300. The instructions may be formed as one or more code modules, each for performing one or more particular tasks. The software may also be divided into two separate parts, in which a first part and the corresponding code modules performs the described methods and a second part and the corresponding code modules manage a user interface between the first part and the user. The software may be stored in a computer readable medium, including the storage devices described below, for example. The software is loaded into the computer system 1300 from the computer readable medium, and then executed by the computer system 1300. A computer readable medium having such software or computer program recorded on it is a computer program product. The use of the computer program product in the computer system 1300 preferably affects an advantageous apparatus for forming a simulation of a proposed training regime and providing a probability risk factor prediction in respect of one or more participants participating in the proposed training regime.

With reference to FIG. 13, an exemplary computing device 1300 is illustrated. The exemplary computing device 1300 can include, but is not limited to, one or more central processing units (CPUs) 1301 comprising one or more processors 1302, a system memory 1303, and a system bus 1304 that couples various system components including the system memory 1303 to the processing unit 1301. The system bus 1304 may be any of several types of bus structures including a memory bus or memory controller, a peripheral bus, and a local bus using any of a variety of bus architectures.

The computing device 1300 also typically includes computer readable media, which can include any available media that can be accessed by computing device 1300 and includes both volatile and non-volatile media and removable and non-removable media. By way of example, and not limitation, computer readable media may comprise computer storage media and communication media. Computer storage media includes media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by the computing device 1300. Communication media typically embodies computer readable instructions, data structures, program modules or other data in a modulated data signal such as a carrier wave or other transport mechanism and includes any information delivery media. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 1303 includes computer storage media in the form of volatile and/or non-volatile memory such as read only memory (ROM) 1305 and random access memory (RAM) 1306. A basic input/output system 1307 (BIOS), containing the basic routines that help to transfer information between elements within computing device 1300, such as during start-up, is typically stored in ROM 1305. RAM 1306-typically contains data and/or program modules that are immediately accessible to and/or presently being operated on by processing unit 1301. By way of example, and not limitation, FIG. 13 illustrates an operating system 1308, other program modules 1309, and program data 1310.

The computing device 1300 may also include other removable/non-removable, volatile/non-volatile computer storage media. By way of example only, FIG. 13 illustrates a hard disk drive 1311 that reads from or writes to non-removable, non-volatile magnetic media. Other removable/non-removable, volatile/non-volatile computer storage media that can be used with the exemplary computing device include, but are not limited to, magnetic tape cassettes, flash memory cards, digital versatile disks, digital video tape, solid state RAM, solid state ROM, and the like. The hard disk drive 1311 is typically connected to the system bus 1304 through a non-removable memory interface such as interface 1312.

The drives and their associated computer storage media discussed above and illustrated in FIG. 13, provide storage of computer readable instructions, data structures, program modules and other data for the computing device 1300. In FIG. 13, for example, hard disk drive 1311 is illustrated as storing an operating system 13YY, other program modules 1314, and program data 1315. Note that these components can either be the same as or different from operating system 1308, other program modules 1309 and program data 1310. Operating system 13YY, other program modules 1314 and program data 1315 are given different numbers hereto illustrate that, at a minimum, they are different copies.

The computing device also includes one or more input/output (I/O) interfaces 1330 connected to the system bus 1304 including an audio-video interface that couples to output devices including one or more of a video display 1334 and loudspeakers 1335. Input/output interface(s) 1330 also couple(s) to one or more input devices including, for example a mouse 1331, keyboard 1332 or touch sensitive device 1333 such as for example a smartphone or tablet device.

Of relevance to the descriptions below, the computing device 1300 may operate in a networked environment using logical connections to one or more remote computers. For simplicity of illustration, the computing device 1300 is shown in FIG. 13 to be connected to a network 1320 that is not limited to any particular network or networking protocols, but which may include, for example Ethernet, Bluetooth or IEEE 802.X wireless protocols. The logical connection depicted in FIG. 13 is a general network connection 1321 that can be a local area network (LAN), a wide area network (WAN) or other network, for example, the internet. The computing device 1300 is connected to the general network connection 1321 through a network interface or adapter 1322 which is, in turn, connected to the system bus 1304. In a networked environment, program modules depicted relative to the computing device 1300, or portions or peripherals thereof, may be stored in the memory of one or more other computing devices that are communicatively coupled to the computing device 1300 through the general network connection 1321. It will be appreciated that the network connections shown are exemplary and other means of establishing a communications link between computing devices may be used.

### Interpretation

In Accordance with

As described herein, ‘in accordance with’ may also mean ‘as a function of’ and is not necessarily limited to the integers specified in relation thereto.

### Composite Items

As described herein, ‘a computer implemented method’ should not necessarily be inferred as being performed by a single computing device such that the steps of the method may be performed by more than one cooperating computing devices.

Similarly objects as used herein such as ‘web server’, ‘server’, ‘client computing device’, ‘computer readable medium’ and the like should not necessarily be construed as being a single object, and may be implemented as a two or more objects in cooperation, such as, for example, a web server being construed as two or more web servers in a server farm cooperating to achieve a desired goal or a computer readable medium being distributed in a composite manner, such as program code being provided on a compact disk activatable by a license key downloadable from a computer network.

### Bus

In the context of this document, the term “bus” and its derivatives, while being described in a preferred embodiment as being a communication bus subsystem for interconnecting various devices including by way of parallel connectivity such as Industry Standard Architecture (ISA), conventional Peripheral Component Interconnect (PCI) and the like or serial connectivity such as PCI Express (PCIe), Serial Advanced Technology Attachment (Serial ATA) and the like, should be construed broadly herein as any system for communicating data.

### Database

In the context of this document, the term “database” and its derivatives may be used to describe a single database, a set of databases, a system of databases or the like which may be stored on an appropriate computer-accessible storage medium or storage device. The system of databases may comprise a set of databases wherein the set of databases may be stored on a single implementation or span across multiple implementations. The term “database” is also not limited to refer to a certain database format rather may refer to any database format. For example, database formats may include MySQL, MySQLi, XML or the like.

### Wireless

The invention may be embodied using devices conforming to other network standards and for other applications, including, for example other WLAN standards and other wireless standards. Applications that can be accommodated include IEEE 802.11 wireless LANs and links, and wireless Ethernet.

In the context of this document, the term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some embodiments they might not. In the context of this document, the term “wired” and its derivatives may be used to describe circuits, devices, systems, methods, techniques, communications channels, etc., that may communicate data through the use of modulated electromagnetic radiation through a solid medium. The term does not imply that the associated devices are coupled by electrically conductive wires.

### Processes

Unless specifically stated otherwise, as apparent from the following discussions, it is appreciated that throughout the specification discussions utilizing terms such as “processing”, “computing”, “calculating”, “determining”, “analysing” or the like, refer to the action and/or processes of a computer or computing system, or similar electronic computing device, that manipulate and/or transform data represented as physical, such as electronic, quantities into other data similarly represented as physical quantities.

### Processor

In a similar manner, the term “processor” may refer to any device or portion of a device that processes electronic data, e.g., from registers and/or memory to transform that electronic data into other electronic data that, e.g., may be stored in registers and/or memory. A “computer” or a “computing device” or a “computing machine” or a “computing platform” may include one or more processors.

The methodologies described herein are, in one embodiment, performable by one or more processors that accept computer-readable (also called machine-readable) code containing a set of instructions that when executed by one or more of the processors carry out at least one of the methods described herein. Any processor capable of executing a set of instructions (sequential or otherwise) that specify actions to be taken are included. Thus, one example is a typical processing system that includes one or more processors. The processing system further may include a memory subsystem including main RAM and/or a static RAM, and/or ROM.

### Computer-Readable Medium

Furthermore, a computer-readable carrier medium may form, or be included in a computer program product. A computer program product can be stored on a computer usable carrier medium, the computer program product comprising a computer readable program means for causing a processor to perform a method as described herein.

### Networked or Multiple Processors

In alternative embodiments, the one or more processors operate as a standalone device or may be connected, e.g., networked to other processor(s), in a networked deployment, the one or more processors may operate in the capacity of a server or a client machine in server-client network environment, or as a peer machine in a peer-to-peer or distributed network environment. The one or more processors may form a web appliance, a network router, switch or bridge, or any machine capable of executing a set of instructions (sequential or otherwise) that specify actions to be taken by that machine.

Note that while some diagram(s) only show(s) a single processor and a single memory that carries the computer-readable code, those in the art will understand that many of the components described above are included, but not explicitly shown or described in order not to obscure the inventive aspect. For example, while only a single machine is illustrated, the term “machine” shall also be taken to include any collection of machines that individually or jointly execute a set (or multiple sets) of instructions to perform any one or more of the methodologies discussed herein.

### Additional Embodiments

Thus, one embodiment of each of the methods described herein is in the form of a computer-readable carrier medium carrying a set of instructions, e.g., a computer program that are for execution on one or more processors. Thus, as will be appreciated by those skilled in the art, embodiments of the present invention may be embodied as a method, an apparatus such as a special purpose apparatus, an apparatus such as a data processing system, or a computer-readable carrier medium. The computer-readable carrier medium carries computer readable code including a set of instructions that when executed on one or more processors cause a processor or processors to implement a method. Accordingly, aspects of the present invention may take the form of a method, an entirely hardware embodiment, an entirely software embodiment or an embodiment combining software and hardware aspects. Furthermore, the present invention may take the form of carrier medium (e.g., a computer program product on a computer-readable storage medium) carrying computer-readable program code embodied in the medium.

### Carrier Medium

The software may further be transmitted or received over a network via a network interface device. While the carrier medium is shown in an example embodiment to be a single medium, the term “carrier medium” should be taken to include a single medium or multiple media (e.g., a centralized or distributed database, and/or associated caches and servers) that store the one or more sets of instructions. The term “carrier medium” shall also be taken to include any medium that is capable of storing, encoding or carrying a set of instructions for execution by one or more of the processors and that cause the one or more processors to perform any one or more of the methodologies of the present invention. A carrier medium may take many forms, including but not limited to, non-volatile media, volatile media, and transmission media.

### Implementation

It will be understood that the steps of methods discussed are performed in one embodiment by an appropriate processor (or processors) of a processing (i.e., computer) system executing instructions (computer-readable code) stored in storage. It will also be understood that the invention is not limited to any particular implementation or programming technique and that the invention may be implemented using any appropriate techniques for implementing the functionality described herein. The invention is not limited to any particular programming language or operating system.

### Means for Carrying Out a Method or Function

Furthermore, some of the embodiments are described herein as a method or combination of elements of a method that can be implemented by a processor or a processor device, computer system, or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

### Connected

Similarly, it is to be noticed that the term connected, when used in the claims, should not be interpreted as being limitative to direct connections only. Thus, the scope of the expression a device A connected to a device B should not be limited to devices or systems wherein an output of device A is directly connected to an input of device B. It means that there exists a path between an output of A and an input of B which may be a path including other devices or means. “Connected” may mean that two or more elements are either in direct physical or electrical contact, or that two or more elements are not in direct contact with each other but yet still co-operate or interact with each other.

### Embodiments

Reference throughout this specification to “one embodiment”, “an embodiment”, “one arrangement” or “an arrangement” means that a particular feature, structure or characteristic described in connection with the embodiment/arrangement is included in at least one embodiment/arrangement of the present invention. Thus, appearances of the phrases “in one embodiment/arrangement” or “in an embodiment/arrangement” in various places throughout this specification are not necessarily all referring to the same embodiment/arrangement, but may. Furthermore, the particular features, structures or characteristics may be combined in any suitable manner, as would be apparent to one of ordinary skill in the art from this disclosure, in one or more embodiments/arrangements.

Similarly it should be appreciated that in the above description of example embodiments/arrangements of the invention, various features of the invention are sometimes grouped together in a single embodiment/arrangement, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment/arrangement. Thus, the claims following the Detailed Description are hereby expressly incorporated into this Detailed Description, with each claim standing on its own as a separate embodiment/arrangement of this invention.

Furthermore, while some embodiments/arrangements described herein include some but not other features included in other embodiments/arrangements, combinations of features of different embodiments/arrangements are meant to be within the scope of the invention, and form different embodiments/arrangements, as would be understood by those in the art. For example, in the following claims, any of the claimed embodiments/arrangements can be used in any combination.

### Specific Details

In the description provided herein, numerous specific details are set forth. However, it is understood that embodiments of the invention may be practiced without these specific details. In other instances, well-known methods, structures and techniques have not been shown in detail in order not to obscure an understanding of this description.

### Terminology

In describing the preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar technical purpose. Terms such as “forward”, “rearward”, “radially”, “peripherally”, “upwardly”, “downwardly”, and the like are used as words of convenience to provide reference points and are not to be construed as limiting terms.

### Different Instances of Objects

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

### Comprising and Including

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: “including” or “which includes” or “that includes” as used herein is also an open term that also means “including at least” the elements/features that follow the term, but not excluding others. Thus, including is synonymous with and means comprising.

### Scope of Invention

Thus, while there has been described what are believed to be the preferred arrangements of the invention, those skilled in the art will recognize that other and further modifications may be made thereto without departing from the spirit of the invention, and it is intended to claim all such changes and modifications as fall within the scope of the invention. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

### INDUSTRIAL APPLICABILITY

It is apparent from the above, that the arrangements described are applicable to the mobile device industries, specifically for methods and systems for distributing digital media via mobile devices.

It will be appreciated that the methods/apparatus/devices/systems described/illustrated above at least substantially provide systems and methods system for forming a simulation of a proposed training regime and providing a probability risk factor prediction in respect of one or more participants participating in the proposed training regime.

The systems and methods disclosed herein, and/or shown in the drawings, are presented by way of example only and are not limiting as to the scope of the invention. Unless otherwise specifically stated, individual aspects and components of the systems and methods disclosed herein may be modified, or may have been substituted therefore known equivalents, or as yet unknown substitutes such as may be developed in the future or such as may be found to be acceptable substitutes in the future. The systems and methods disclosed herein may also be modified for a variety of applications while remaining within the scope and spirit of the claimed invention, since the range of potential applications is great, and since it is intended that the present systems and methods disclosed herein be adaptable to many such variations.

The claims defining the invention are as follows:

1. A system for forming a simulation of a proposed training regime and providing a probability risk factor prediction in respect of one or more participants participating in said proposed training regime; the system comprising:
a user input means adapted for accepting user input regarding:
a proposed training regime; and
training data regarding one or more participants of the proposed training regime;
a storage means adapted for storing records regarding:
said historical training data regarding one or more past training activity;
historical participant data regarding one or more participants of the proposed training regime; and
historical actual participant outcomes from said past training activity; and
a system controller comprising a processor adapted to:
receive user input regarding said proposed training regime and proposed participants;
perform calculations to analyse said user input in conjunction with said historical training data and said historical participant outcomes;
perform calculations to compute a probability risk factor prediction for each proposed participant as a result of participating in said proposed training regime; and
display a visual representation of said computed probability risk factor prediction on a user interface.

2. A system as claimed in claim 1 wherein said user input means and said user interface is located on a client device and said system controller is located on a server device in networked communication with said client device.

3. A system as claimed in claim 2 wherein said client device comprises a mobile computing device in wireless networked connected communication with said server device.

4. A system as claimed in claim 1 wherein said probability risk factor prediction comprises a prediction of the risk of injury for each proposed participant as a result of participating in a proposed future training regime.

5. A system as claimed in either claim 1 or claim 2 wherein said proposed training regime comprises a proposed physical exercise plan.

6. A system as claimed in claim 5 wherein said user input regarding said proposed training regime comprises one or more parameters selected from the group of sprint distance, jog distance, running pace, weight of one or more training aids, exercise type, performance metrics.

7. A system as claimed in either claim 5 or claim 6 wherein said user input regarding said one or more participants comprises one or more parameters selected from the group of age, gender, weight, height, previous injury history, previous historical training regime participation outcomes, and other performance and physiological related metadata.

8. A system as claimed in claim 7 wherein where one of said participants has a history of an injury, said historical training data regarding one or more participants of the proposed training regime further comprises an injury modifier which decreases as a function of time from the time of the initial injury such that a calculated prediction of probability risk factor for the participant in a future proposed training regime is increased by the injury modifier for a predetermined time period after the initial injury.

9. A system as claimed in claim 8 wherein said injury modifier decreases with respect to time commencing from the time of the initial injury in accordance with a custom time-based function based on the frequency and/or severity of past participant injuries.

10. A system as claimed in any one of the preceding claims wherein said simulation of said proposed training regime is represented by an artificial neural network adapted to accept input parameters obtained by pre-processing of raw data comprising one or more of:
said historical training and participant data retrieved from said storage means;
participant data received from said input means; and
proposed training regime data received from said input means.

11. A system as claimed in claim 10 wherein said pre-processing of said raw data comprises one or more data processing procedures selected from the group of: data smoothing, data normalisation, data aggregation, data sampling, Synthetic Minority Over-sampling Technique (SMOTE).

12. A system as claimed in either claim 10 or claim 11 wherein said input to said artificial neural network comprise an input vector comprising an aggregation of input parameters over a predetermined time period.

13. A system as claimed in any one of claims 10 to 12 wherein said artificial neural network is adapted to simulate a predicted probability risk factor for each participant member of a team comprising a plurality of participant members, wherein said team is scheduled to participate in a future said proposed training regime.

14. A system as claimed in any one of the preceding claims further comprising means to select a predetermined time window for analysis of participant probability risk factor during a proposed training regime comprising a plurality of training exercises.

15. A system as claimed in claim 14 wherein said prediction of probability risk factor is provided in real time on selection of a predetermined time window indicative of the duration of the proposed training regime.

16. A system as claimed in any one of the preceding claims wherein said prediction of probability risk factor is provided in real time on modifications of said proposed training regime.

17. A system for forming a simulation of a proposed training regime and providing a probability risk factor prediction, said system comprising one or more active artificial neural networks adapted for simulation of one or more participants of a proposed exercise training regime such that said simulation provides outputs comprising a predicted probability risk factor for one or more said participants of said proposed training regime.

18. A system as claimed in claim 17 wherein said system further comprises one or more training artificial neural networks correlated to each of said active artificial neural networks, and wherein:
on receipt of data comprising data in respect of said proposed exercise training regime and/or in respect of one or more of said participants said system is adapted to perform computational training of each said training artificial neural network to provide a predicted probability risk factor; and
wherein in the event said predicted probability risk factor obtained from said training artificial neural network provides a more accurate predicted probability risk factor than that obtained from a corresponding one of said active artificial neural networks, said corresponding said active artificial neural network is replaced by said training artificial neural network to form a new active artificial neural network, and a new training artificial neural network is formed for ongoing training of said artificial neural networks.

19. A method of planning a proposed training regime for one or more training participants, the method comprising the steps of:
entering user input to a system controller, said user input comprising information regarding a proposed training regime and one or more proposed training participants, said system controller comprising
user input means for accepting said user input;
storage means adapted to store records regarding: historical training data; historical training regimes and historical actual participant outcomes from said historical training regimes;
a processor adapted to perform calculations to:
analyse said user input in conjunction with said historical training data and said historical participant outcomes; and
compute a probability risk factor prediction comprising a prediction of the risk of injury for each proposed participant as a result of participating in said proposed training regime;
receiving calculated probability risk factor prediction data including prediction of the risk of injury for each proposed participant as a result of participating in said proposed training regime; and
modifying said proposed training regime to minimise the risk of injury for one or more of the proposed participants.

20. A computer-readable medium storing computer-executable instructions that when executed by a computer cause the computer to perform a method for planning a proposed training regime for one or more training participants, said computer comprising a user input means; storage means; and a processor;
wherein said method comprises the steps of:
receiving user input comprising information regarding a proposed training regime and one or more proposed training participants;
effecting the processor to retrieve records from a storage means, said records comprising historical training data, historical training regimes and historical actual participant outcomes from said historical training regimes;
effecting the processor to perform calculations to analyse said user input in conjunction with said historical training data and said historical participant outcomes;
effecting the processor to perform calculations to compute a probability risk factor prediction comprising a prediction of the risk of injury for one or more of said proposed participant as a result of participating in said proposed training regime; and
displaying a visual representation of said computed probability risk factor prediction on a user interface.

21. A computer-readable medium storing computer-executable instructions as claimed in claim 20 wherein said method further comprises accepting user input with respect to the displayed visual representation, said user input comprising a selection of a time window with respect to said proposed training regime,
wherein, upon receipt of said user input, said processor is effecter to perform calculations to compute a probability risk factor prediction for at least one or more participants at the end of the selected time window.

22. A computer program product having a computer readable medium having a computer program recorded therein for forming a simulation of a proposed training regime and providing a probability risk factor prediction for one or more proposed participants of said proposed training regime, said computer comprising a user input means; storage means; and a processor; said computer program product comprising:
computer program code means for receiving user input comprising information regarding a proposed training regime and one or more proposed training participants;
computer program code means for effecting a processor to retrieve records from a storage means, said records comprising historical training data; and historical training regimes and historical actual participant outcomes from said historical training regimes;
computer program code means for effecting a processor to perform calculations to analyse said user input in conjunction with said historical training data and said historical participant outcomes;
computer program code means for effecting a processor to perform calculations to compute a probability risk factor prediction comprising a prediction of the risk of injury for one or more of said proposed participant as a result of participating in said proposed training regime; and
computer program code means for displaying a visual representation of said computed probability risk factor prediction on a user interface.

23. A computer program for forming a simulation of a proposed training regime and providing a probability risk factor prediction for one or more proposed participants of said proposed training regime, said program comprising:
code for receiving user input comprising information regarding a proposed training regime and one or more proposed training participants;
code for effecting a processor to retrieve records from a storage means, said records comprising historical training data; and historical training regimes and historical actual participant outcomes from said historical training regimes;
code for effecting a processor to perform calculations to analyse said user input in conjunction with said historical training data and said historical participant outcomes;
code for effecting a processor to perform calculations to compute a probability risk factor prediction comprising a prediction of the risk of injury for one or more of said proposed participant as a result of participating in said proposed training regime; and
code for displaying a visual representation of said computed probability risk factor prediction on a user interface.

24. A system comprising:
one or more processors;
memory coupled to the one or more processors and configured to store instructions, which, when executed by the one or more processors, causes the processors to perform operations comprising:
receiving user input comprising information regarding a proposed training regime and one or more proposed training participants;
effecting a processor to retrieve records from a storage means, said records comprising historical training data, historical training regimes and historical actual participant outcomes from said historical training regimes;
effecting a processor to perform calculations to analyse said user input in conjunction with said historical training data and said historical participant outcomes;
effecting a processor to perform calculations to compute a probability risk factor prediction comprising a prediction of the risk of injury for one or more of said proposed participant as a result of participating in said proposed training regime; and
displaying a visual representation of said computed probability risk factor prediction on a user interface.

25. A non-transitory storage device storing instructions that, when executed by one or more processors, cause the one or more processors to perform operations comprising:
receiving user input comprising information regarding a proposed training regime and one or more proposed training participants;
effecting a processor to retrieve records from a storage means, said records comprising historical training data; and historical training regimes and historical actual participant outcomes from said historical training regimes;
effecting a processor to perform calculations to analyse said user input in conjunction with said historical training data and said historical participant outcomes;
effecting a processor to perform calculations to compute a probability risk factor prediction comprising a prediction of the risk of injury for one or more of said proposed participant as a result of participating in said proposed training regime; and
displaying a visual representation of said computed probability risk factor prediction on a user interface.

26. A computer program product for simulating a proposed training regime and providing a probability risk factor prediction, the computer program product comprising a computer readable storage medium having program code embodied therewith, the program code executable by a processor to:
receive user input comprising information regarding a proposed training regime and one or more proposed training participants;
effect the processor to retrieve records from a storage means, said records comprising historical training data; and historical training regimes and historical actual participant outcomes from said historical training regimes;
train statistical models in respect of simulating said training regime and each of said participants of said proposed training regime, said training of said statistical models comprising:
effecting a processor to perform calculations to analyse said user input in conjunction with said historical training data and said historical participant outcomes; and
effecting a processor to perform calculations to compute a probability risk factor prediction comprising a prediction of the risk of injury for one or more of said proposed participant as a result of participating in said proposed training regime;
compute a probability risk factor prediction with respect to at least one or more of said participants; and
display a visual representation of each said computed probability risk factor prediction on a user interface.
